(19) United States
(12) Patent Application Publication
(10) Pub. No.: US 2021/0154351 A1
(43) Pub. Date: May 27, 2021

(54) PSYLLIUM BASED MOISTURE ABSORBENT MATERIAL

(71) Applicant: Xiros Limited, Leeds, Yorkshire (GB)

(72) Inventors: Christopher Ochayi Agboh, Leeds (GB); David Farrar, Leeds (GB)

(21) Appl. No.: 16/641,608

(22) PCT Filed: Aug. 23, 2018
(86) PCT No.: PCT/GB2018/052392

§ 371 Date: Feb. 24, 2020

(30) Foreign Application Priority Data
Aug. 24, 2017 (GB) 1713623.5

(51) Int. Cl. A61L 15/40; A61F 13/475; A61L 15/28; A61L 15/42; A61L 26/00; C08L 5/14

(52) U.S. Cl. CPC A61L 15/40; A61F 13/4753; A61L 15/28; A61L 15/425; A61L 2400/04; A61L 26/0085; C08L 5/14; A61L 2300/102; A61L 2300/212; A61L 26/0023

(57) ABSTRACT

A moisture absorbent material capable of biomedical application to absorb and manage bodily fluids such as blood and wound exudate as a wound or first aid dressing or bandage. The material is a psyllium foam based material offering high absorbency and potentially haemostatic characteristics.

### FIELD OF INVENTION

The present invention relates to a moisture absorbent psyllium foam and in particular, although not exclusively, to a psyllium foam based material suitable for use as a wound dressing and in particular a homeostatic dressing.

### BACKGROUND ART

Polysaccharides, due their non-toxic, biocompatible and biodegradable properties have found application as wound dressings, drug delivery agents and other healthcare materials. Psyllium husk is a natural polysaccharide (also known as ispaghula) and is a typically obtained from Plantago ovata. Psyllium is a partially water soluble, hydrophilic material and has considerable swelling capability in contact with water.

U.S. Pat. No. 5,204,103 discloses a psyllium based wound treatment material in which psyllium particulates are combined with other active substances such as enzymes and antiseptic agents to promote wound healing.

WO 2005/086697 discloses the use of psyllium seed gum for swellable devices to occlude blood vessels or act as a tamponade for nasal or other bleeds. Other uses of psyllium particles and fibres within the healthcare sector are described in WO 96/00094 and WO 1998/001167.

However, existing psyllium containing materials offer limited moisture absorbency and retention. Additionally, conventional wound dressings are susceptible to decomposition particularly during extended contact periods with a wound. Accordingly, what is required is a biocompatible moisture absorbent material that addresses these problems.

### SUMMARY OF THE INVENTION

It is an objective of the present invention to provide a moisture absorbent material configured to absorb and maintain a high level of an absorbed fluid. It is a specific objective to provide a moisture absorbent material for the biomedical field to absorb and retain bodily fluids.

It is a further specific objective to provide a moisture absorbent material suitable for use as wound and first aid dressings, bandages and in particular haemostatic bandages or dressings for intra surgical or external use, veterinary poultices, hygiene articles, sanitary pads, liquid filtration mediums and food packaging.

The objectives are achieved by providing a psyllium based foam material offering high moisture absorbency and retention. The present psyllium based foams are also configured with the desired physical and mechanical characteristics as they absorb and manage bodily fluids so as to not degrade or degenerate on moisture uptake. The present foams are further beneficial to provide desired moisture vapour transmission across the foam material due, in part, to the porosity of the foam. Moreover, the present foams according to specific implementations exhibit desired flexibility, softness and skin contact adhesion and release (commonly referred to as tack). Such characteristics are advantageous to enable the material to conform to the body on initial placement and during fluid absorption in addition to minimising or eliminating skin maceration that would otherwise be associated with highly adhesive or ‘sticky’ materials when exposed to bodily fluids such as blood and wound exudate. Accordingly, the present fluid absorbing materials are capable of internal and external body usage and optionally to provide haemostatic characteristics to greatly facilitate wound healing and repair.

According to a first aspect of the present invention there is provided a moisture absorbent material comprising: a psyllium foam.

The present invention is formed as a foam and in particular a non-crosslinked foam. As may be appreciated, crosslinking is a well-known technique to reduce absorbency of polymers by restricting molecular motion of the polymer chains. The present foam is accordingly configured for expansion between the polymer chains so as to be capable of swelling and hence comprise high moisture absorbency characteristics.

Optionally, the psyllium foam is compositionally a main component of the material by 20 wt %. Optionally, the absorbent material comprises psyllium foam in at least 50 wt %, at least 60 wt %, at least 70 wt %, at least 80 wt %, at least 90 wt %, at least 95 wt %, at least 96 wt %, at least 97 wt %, at least 98 wt % or at least 99 wt %. Such a configuration has been found to maximise the fluid absorption and retention characteristics of the material comprising exclusively or almost exclusively psyllium foam.

The moisture absorbent foam is porous and may comprise an average pore size in a range 50 μm to 2 mm; 100 μm to 2 mm; 200 μm to 2 mm; 300 μm to 2 mm; 400 μm to 2 mm; 500 μm to 2 mm; 600 μm to 2 mm; 700 μm to 2 mm; 800 μm to 2 mm; 50 μm to 1.5 mm; 50 μm to 1 mm; 50 μm to 800 μm; 100 μm to 800 μm; 200 μm to 800 μm; 300 μm to 800 μm; 400 μm to 800 μm; 50 μm to 600 μm; or 200 μm to 600 μm. This average pore size has been found to provide the desired physical and mechanical characteristics with regard to absorbency, retention, integrity, moisture vapour transmission, flexibility, softness and tack. Pore size has been identified specifically to achieve the desired absorbency and moisture vapour transmission across the material. Preferably, the present material comprises an average pore size of greater than 150 μm or 200 μm or even 300 μm. As may be appreciated, smaller pore sizes can provide less absorbency as less fluid can be accommodated within the pores. Additionally, smaller pores may have a tendency to become blocked by debris in the wound e.g. blood, cellular debris and/or necrotic tissues. The present pore size additionally provides a macrostructure that is maintained with liquid absorption capability that does not collapse due to swelling. In particular, the present foams maintain integrity to provide easy removal when used as wound dressing.

The present invention provides a highly absorbent material for use in applications where high absorbency is important specifically including wound dressings. The present psyllium-based foam is configured to absorb liquid according to at least two modes. In a first mode, liquid is absorbed by the polymer itself and according to a second mode the liquid is absorbed by the pores of the foam. The present foam via its pore size and other physical and mechanical and chemical characteristics as described herein provides a highly absorbent material that is preferably not adapted for tissue ingrowth. That is, the present foam provides an absorbent material and not a scaffold for the ingrowth of tissue and cells.

Optionally, the psyllium foam is derived from psyllium husk and optionally Plantago Ovata. Optionally, the psyllium foam may be derived from any one or a combination of Plantago Ovata; Plantago major; P. psyllium; P. amplexicauli; P. asiatica; P. lanceolate; P. insularis.

Optionally, the material may comprise any one or a combination of: hydrogen peroxide; a surfactant; lecithin. Hydrogen peroxide is advantageous to control microbial growth, and to contribute to any haemostatic characteristics in addition to providing a colourless white foam. The hydrogen peroxide and/or the surfactant may also act to increase foaming and accordingly control the creation of pore size and the foam/pore density. Lecithin is advantageous to facilitate cell growth and regeneration.

Optionally, the material may comprise any one or a combination of: a polysaccharide (to increase integrity and absorbency); a polysaccharide based material (to increase integrity and absorbency); a hydrocolloid forming compound; an alginate (to increase integrity and absorbency); chitosan (to increase integrity and absorbency in addition to providing antimicrobial characteristics); chitin (to increase integrity and absorbency in addition to imparting antimicrobial characteristics and material strength); pectin (to increase integrity and absorbency); carboxymethyl cellulose (to increase integrity and absorbency); hydroxylpropyl methylcellulose (to increase integrity and absorbency); gellan (to increase integrity and absorbency); konjac (to increase integrity and absorbency).

Optionally, the material may further comprise any one or a combination of: a protein; silk; gelatin; collagen; keratin. Such compounds are advantageous to increase the strength of the material, to facilitate cell growth and regeneration at a wound site and to increase the integrity of the material to avoid degeneration during uptake of moisture.

Optionally, the material may further comprise any one or a combination of: a synthetic polymer; polyethylene oxide; polyacrylic acid; acetic acid; polyacrylate; polyacrylonitrile; polyvinyl alcohol; a polyol; glycerol; a glycol; a diol; an alkaline glycol; propylene glycol. Such additives are advantageous to increase the flexibility, elasticity, integrity and absorption of the material. Polyacrylic acid is further beneficial to increase the absorbency of the material.

Optionally, the material may further comprise any one or a combination of: calcium chloride hydrate: calcium chloride dehydrate: ferric sulphate hydrate; ferric sulphate dehydrate. Such hydrates may be added to increase the haemostatic characteristics of the material to facilitate blood clotting and hence wound healing and repair.

Optionally, the material may further comprise any one or a combination of: a drug (for example a painkiller and/or an anti-inflammatory); an enzyme (for example a platelet-derived growth factor); a growth enhancer; a living cell (for example fibroblast); an antimicrobial (for example an antibiotic); a metal based antimicrobial; an antimicrobial agent metal ion selected from any one or a combination of: Ag, Zn, Cu, Ti, Pt, Pd, Bi, Sn, Sb.

According to specific implementations of the present invention there is provided a wound dressing, a haemostat, a hygiene article, a mammalian sanitary pad, a liquid filtration medium, or a food packaging material as claimed herein.

According to a further aspect of the present invention there is provided a method of manufacturing a moisture absorbent material comprising: forming a psyllium gel from an aqueous psyllium solution; and creating a psyllium foam from the psyllium gel.

Optionally, the step of creating the psyllium foam from the psyllium gel comprises freeze drying the psyllium gel. As will be appreciated, freeze drying via sublimation is effective to create the foam structure resulting from the formation of ice crystals within the gel during freeze drying.

Optionally, the step of creating the psyllium foam from the psyllium gel comprises agitating the psyllium gel to create air bubbles to form the psyllium foam and drying the psyllium foam. Agitation of the gel may be provided by vigorous stirring or blending.

Optionally, the step of creating the psyllium foam from the psyllium gel comprises adding a foaming agent to the psyllium solution and/or psyllium gel and drying the psyllium foam. The foaming agents may be added to the psyllium solution and/or the psyllium gel optionally in addition to agitating the gel. Optionally, the foaming agent may comprise any one or a combination of the following: hydrogen peroxide; a surfactant; lecithin.

Optionally, the method may comprise adding one or more additives (as described herein) to the psyllium solution or gel.

### BRIEF DESCRIPTION OF DRAWINGS

Specific implementations of the present invention will now be described, by way of example only, and with reference to the accompanying drawings in which:

FIG. 1 is a microscope image of an absorbent material according to example 2 described herein;

FIG. 2 is a microscope image of an absorbent material according to example 3 described herein;

FIG. 3 is a microscope image of an absorbent material according to example 8 described herein;

FIG. 4 is a microscope image of an absorbent material according to example 10 described herein;

FIG. 5 is a microscope image of an absorbent material according to example 13 described herein;

FIG. 6 is a microscope image of an absorbent material according to example 14 described herein.

### DETAILED DESCRIPTION OF OPTIONAL EMBODIMENTS OF THE INVENTION

Absorbent materials within the biomedical fields must be capable of absorbing and managing bodily fluids such as blood and wound exudates. Specific implementations of the present invention include materials and products for use as wound dressings, first aid dressings, haemostatic dressings and bandages for intra surgical or external use, veterinary poultices, baby diapers, mammalian sanitary pads and other hygiene articles. Specific implementations of the present invention further include food packaging materials.

In addition to absorbing and retaining high levels of bodily fluids, the present biomedical materials are biocompatible to avoid irritation of the body region with which the material is in contact. Moreover, the present materials are configured in certain configurations to provide haemostatic and soothing properties to facilitate wound healing and repair.

The inventors have identified psyllium based materials optionally derived from Plantago ovata as particularly advantageous for biomedical applications when formulated as a foam in contrast to particulate and spun fibre based constructions. That is, according to specific implementations of the present invention, a biomedical psyllium-foam based material may be formed exclusively from psyllium i.e. substantially 100 wt % or at least 95 wt/o psyllium. Optionally, the psyllium foam based material may comprise additional components such as alginates, chitosan, chitin, pectin, carboxymethyl cellulose (CMC) and proteins, silk, gelatin, collagen etc. In particular, the inventors have discovered that Psyllium foam, in contrast to the alternative fibre or particulate format, is highly absorbent and structurally robust as a self-supporting and free standing structure i.e., when used directly as a wound dressing, pad, healthcare material or other absorbent product. The present foams may include high proportions of psyllium (up to 100%) which would otherwise be impossible to spin into fibres where it is noted that conventionally psyllium is required to be combined with other materials such as alginates in order to make fibre based materials.

The following example psyllium foam based materials were prepared and various physical and mechanical properties of the end foams observed and assessed for suitability as a biomedical absorbent products. In particular, moisture absorbency and retention were investigated by material immersion in pre-prepared solutions in addition to in-vitro haemostasis testing by direct measurement of clotting time.

Table 1 summarises the 18 material composition examples based on psyllium foam identifying optional foam additives at their respective concentrations.

TABLE 1A
examples 1 to 9 psyllium foam preparations

| | Examples 1-9 | | | | | | | | |
|---|---|---|---|---|---|---|---|---|---|
| Composition (%) | 1 | 2 | 3 | 4 | 5 | 6 | 7 | 8 | 9 |
| Psyllium (filtered) (P) | 1.2 | ~2 | | | ~1.0 | 1.2 | | | |
| Psyllium (unfiltered) (P) | | | 2 | 5 | | | 2.5 | 1.2 | 1.5 |
| RO water | 98.8 | 95 | 95 | 92 | ~96 | 95.7 | 97.3 | 64.2 | 95.2 |
| Tween 20 | | 1 | 1 | 1 | | | | | 0.5 |
| H₂O₂ | | 2 | 2 | 2 | 0.9 | 2 | 0.10 | | 2 |
| CaCl₂ dihydrate (ca) | | | | | | 0.01 | | | |
| Glycerol (Gly) | | | | | 1.8 | | 0.10 | | |
| Lecithin (LE) | | | | | 0.26 | 0.08 | | | |
| Propylene glycol (PG) | | | | | | 1 | 0.03 | | |
| 2% acetic acid | | | | | | | | 34.0 | |
| Chitosan (CS) | | | | | | | | 0.6 | |
| Pectin (PE) | | | | | | | | | 0.8 |

TABLE 1B
examples 10 to 18a (18b) psyllium foam preparations

| | Examples 10-18b | | | | | | | | |
|---|---|---|---|---|---|---|---|---|---|
| Composition | | | | | | | | | 18a |
| (%) | 10 | 11 | 12 | 13 | 14 | 15 | 16 | 17 | (18b) |
| Psyllium | | | | | | | | | |
| (filtered) | | | | | | | | | |
| (P) | | | | | | | | | |
| Psyllium | 1.5 | 2 | 2 | 1.5 | 1.5 | 1 | 2.4 | 1.5 | 3 |
| (unfiltered) | | | | | | | | | (2) |
| (P) | | | | | | | | | |
| RO water | 94.7 | 94 | 96 | | 97.7 | | 96 | 96 | 90 |
| | | | | | | | | | (92) |
| Tween 20 | 0.5 | | | 0.5 | | | | 0.5 | 1 |
| H₂O₂ | 2 | | | 2 | | | | | (5) |
| CaCl₂ | 0.5 | | | | | | 0.04 | | 1 |
| dihydrate (ca) | | | | | | | | | |
| Glycerol | | | | | | | | | (1) |
| (Gly) | | | | | | | | | |
| Lecithin (LE) | | | | | | 0.08 | 0.08 | | |
| Propylene | | | | | | 2 | 5 | | 5 |
| glycol (PG) | | | | | | | | | |
| Pectin (PE) | 0.8 | | | | | | | | |
| Gelatin | | 2 | | | | | | | |
| (Porcine) | | | | | | | | | |
| (GeP) | | | | | | | | | |
| Gelatin | | | 2 | 0.8 | | | | | |
| (bovine) | | | | | | | | | |
| (GeB) | | | | | | | | | |
| Carboxymethyl | | | | | 0.80 | | 0.60 | | |
| cellulose | | | | | | | | | |
| (CMC) | | | | | | | | | |
| Fe₂(SO4)₃ | | | | | | | 0.08 | | |
| dihydrate | | | | | | | | | |
| Alginates: | | | | | | 0.5 | 0.60 | | |
| HM | | | | | | | 0.20 | | |
| HG | | | | | | | | | |
| Reformed | | | | | | | | 0.4 | |
| silk fibre | | | | | | | | | |
| (SF) | | | | | | | | | |
| Lithium | | | | | | | | 1.6 | |
| Bromide | | | | | | | | | |

### Example 1: Preparation of Psyllium Foam from a Filtered Psyllium Gel Solution

First, 7.5 g of psyllium husk (Frøskaller) corresponding to a 2.5% w/w concentration was mixed gradually using a Philips HR2074 blender. The powder was added gradually through the opening in the lid into already stirring RO water (292.5 g) contained in the blender glass jar. The mixture was stirred continuously at the minimum speed for 1 minute and then transferred into a plastic beaker for the glass jar to be cleaned and for the filter (˜100 μm sieve size) and jar lid to be installed for the filtration process. The mixture was then poured into the filter and the blender run at the maximum speed for 3 minutes. The filtered gel (referred to as filtrate 1) was very viscous and difficult to pour out of the jar with the lid on. The lid was therefore removed and with the measuring cup on the filter, the blender jar was tilted and the filtered gel scooped out of the jar into a cleaned vessel. The semi-solid gel residue left in the filter was poured back into the blender jar and mixed vigorously with 300-350 g fresh RO water and filtered as described above to give filtrate 2. Filtrates 1 and 2 were transferred into the blender jar and mixed at maximum speed to introduce as much air bubbles as possible in the final foam. The final concentration of psyllium (1.2%) in the foam was determined from a known weight of the foam (80 g) dried to constant weight in an oven at 105° C.

The was foam was shared into glass petri dishes in weights between 60 and 100 g, placed in a fridge overnight (˜16 h) and then stored in a freezer (−21° C.) for 24 h before freeze drying using a Biopharma laboratory bench top freeze dryer (condenser temperature −40 to −60° C.; vacuum pressure 265-280 μB; drying chamber ambient) for 35 h. Foams produced were 1-mm thick, white, flexible with soft handle, crystal appearance on the surface and easily compressed.

### Example 2: Preparation of Psyllium Foam from Filtered Psyllium Gel Solution Containing Hydrogen Peroxide and Tween 20

This example was investigated to assess the effect of addition of a surface active agent (Tween 20) and hydrogen peroxide (antimicrobial agent). Here 15 g (3% w/w) of psyllium husk were weighed and stirred briefly (1 minutes) into 485 g RO water using a Philips blender. The mixture was filtered as described in example 1 except that the final residue was further filtered by squeezing manually through a woven plain cloth and collecting the resulting gel extract to give a final filtrate weight of 510 g. The solid content of the filtrate (2% w/w) was determined as in example 1 but using 45 g solution. The remaining fraction 465 g was transferred into the blender jar and after an addition of 4.65 g (1% w/w) Tween 20 to increase air bubbles in the foam, the mixture was mixed for 2 minutes. This was followed by addition of 9.2 g (2% w/w) hydrogen peroxide (H₂O₂) and further mixing until the foam could be poured from the mixer without sticking to the glass jar. The peroxide was added to control microbial growth and to give a whiter foam. Foams were soft and flexible with absorbent characteristics shown in Table 4 produced as described in example 1. But the presence of Tween 20 resulted in slightly heavier integral foam. However, in this example the foam expanded more in the freezer dryer chamber on application of vacuum pressure due mostly to the presence of H₂O₂; so, it was important to ensure that the amount of foam in each dish was not more than 100 g.

### Example 3: Preparation of Psyllium Foam from Unfiltered Psyllium Gel Solution Containing Hydrogen Peroxide and Tween 20

Psyllium solution in water contains various fractions some of which are insoluble particulates (10-15%). This example was investigated to assess the effects, if any, of these insoluble psyllium particulates on the texture and absorbency of foam produced. The foam was prepared by first mixing 10 g (2% w/w) psyllium in 475 g of RO water for 1 minute using a blender. This was followed by the addition of 5 g (1% w/w) Tween 20 and mixing for 1 minute before adding 10 g (2% w/w) H₂O₂ and mixing (for about 3 minutes) until the foam could be removed from the blender jar completely by tilting it. The foam was shared into glass dishes (60-100 g) as outlined in previous examples, stored in the fridge for about 1 hour before removing into a freezer and storing overnight. Freeze drying was carried out as outlined in example 1. The foam produced was white, soft, flexible, compressible, easily bendable but with traces of black specks. In solution, the foam absorbed and held together well. However, as observed in example 2, the foam became less integral after storing in solution for 30 minutes.

### Example 4: Preparation of Psyllium Foam from Unfiltered High Concentration of Psyllium Gel Containing Hydrogen Peroxide and Tween 20

The foam was prepared by weighing 5 g (1% w/w) Tween 20 and 10 g (2% w/w) H₂O₂ into 450 g of RO water contained in a blender jar and adding gradually 25 g (5% w/w) psyllium husk (Frøskaller) whilst being stirred. The blender stopped as the psyllium dissolved, but restarted after briefly mixing manually. Mixing continued until a semi-solid but uniform foam was obtained. This was tipped out of the jar and shared into glass dishes, then pressed into shape using non-stick flat bottomed glass containers. The foams were stored for 30 minutes in a fridge, then in a freezer (−21° C.) for 3 days before freeze drying as described in example 1 but for 48 h. The samples expanded during drying in a similar fashion as observed in examples 2 and 3. The expanded section of the foam was soft, smooth and easily compressible whilst the unexpanded section dried into a very rough and hard foam.

### Example 5: Preparation of Psyllium Foam from Filtered Psyllium Gel Containing Lecithin, Glycerol, Hydrogen Peroxide and Tween 20

Glycerol and lecithin was added to the preparation to impart softness and to change the texture and absorbency of the psyllium foam. The foam was prepared by first mixing 12 g (1.5% w/w) of psyllium into 788 g of RO water and filtering the mixture as described in examples 1 and 2 to give 540 g of filtered psyllium solution with a solid content of about 1% w/w. The psyllium solution was transferred into the blender jar, gently stirred at the minimum speed, whilst the remaining items, 10 g glycerol (1.8% w/w), 1.35 g lecithin (0.26% w/w) and 5 g H₂O₂ (0.9% w/w), calculated on the weight of 540 g psyllium solution, were being added in succession to the stirring solution. Once added, mixing was continued at higher speed for 5 minutes. Then as in previous examples, the foam was shared into dishes, refrigerated for 2 hours, stored in a freezer for 7 days before freeze drying for 40 hours to give a very thin, pale yellow, dense, sticky and highly elastic foam, with the yellow colour being attributed to lecithin.

### Example 6: Preparation of Psyllium Foam from Filtered Psyllium Gel Containing Lecithin, Propylene Glycol, Hydrogen Peroxide and Calcium Salt

In this example propylene glycol was used instead of glycerol. The addition of propylene glycol is intended to reduce loss of moisture and to preserve the foam. The foam was prepared as in example 5 except for a lower concentration of psyllium, lecithin and the introduction of a small amount of calcium chloride dihydrate along with propylene glycol (as shown in Table 1A) to increase absorbency, reduce stickiness and lower foam density whilst preserving the foam wet integrity observed in example 5.

### Example 7: Preparation of Psyllium Foam from Unfiltered Psyllium Gel Containing Glycerol, Propylene Glycol and Hydrogen Peroxide

In this example, propylene glycol and glycerol were added to the foam but with a reduced concentration of glycerol as the very low absorbency observed in example 5 may be due to high glycerol content. The foam was prepared by mixing 0.2 g (0.03% w/w) propylene glycol, 0.72 g (0.1% w/w) glycerol and 0.72 g (0.1% w/w) hydrogen peroxide into 25 g of RO water and adding the mixture gradually into a well stirred psyllium solution which has been prepared by first soaking 17.5 g (2.5% w/w) psyllium husk in 700 g water for 4 hours and mixing. Then, as in previous examples, the foam was shared into dishes, refrigerated for 2 hours, stored in a freezer for 3 days and then freeze dried. The foam produced was dull white with reduced softness (whilst being compressible) and broke on bending.

### Example 8: Psyllium Foam Containing Chitosan

Initially, a 1.71% w/w (3 g) chitosan solution was prepared by dissolving the chitosan powder (from shrimp shells, ≥75% deacetylated—Sigma-Aldrich) in 172 g of 2% acetic acid. The solution was then diluted to 0.6% w/w by adding 325 g of RO water and mixing thoroughly. Then 6.3 g psyllium husk (Frøskaller) corresponding to a 1.24% w/w concentration, were stirred into the solution and mixed until a consistent and thick foam with an overall solid content of 1.84% w/w and pH 3 was formed. The foam as usual was shared into glass dishes, refrigerated for 3 days, and then left in the freezer for 3 days before freeze drying over 2 days. The foam produced was pale yellow, light, flexible, soft, compressible, and bendable without breaking (thickness 3-9 mm).

### Example 9: Psyllium Foam Containing Pectin

Psyllium husks from Frøskaller (7.5 g, 1.5% w/w) was weighed and mixed with low methoxyl pectin (4.1 g, 0.82% w/W) obtained from FMC (Italy S.R.I) with methoxyl (—OCH3) groups content about 10% and galacturonic acids content of about 90%. The mixture was stirred vigorously for 3 minutes before the addition of hydrogen peroxide (10 g, 2% w/w) and Tween 20 (2.5 g, 0.5% w/w) and by further mixing until consistent foam was produced. The foam was transferred into dishes, allowed to cool down for 2 h in a fridge before storing in a freezer overnight. The foam was dried in a freeze dryer over 50 h. The foam obtained was white, flexible, soft and easily compressed.

### Example 10: Psyllium Foam Containing Pectin, Hydrogen Peroxide, Tween 20 and Calcium Chloride Dihydrate

This example was an attempt to improve the psyllium-pectin foam integrity in solution A or saline. The foam was prepared as described in example 9 except for the addition of 2.5 g (0.5% w/w) calcium chloride dihydrate. The foam obtained was white, flexible but with reduced softness but still easily compressible.

### Example 11: Psyllium Foam Containing Gelatin (Type A)

The gelatin powder (10 g, 2% w/w) used was obtained from porcine skin (Type A, gel strength 300) and psyllium husk ((10 g, 2% w/w) from Frøskaller. The two polymers were weighed, mixed together and gradually added to a gently stirring 480 g of RO water contained in a Philips blender. Once the powders had been added, the mixture was stirred continuously and vigorously for 7 minutes before checking for uniformity and temperature (40° C.). This was followed by further stirring and checking (10 min) until the temperature reached 60° C. to ensure that the gelatin was properly dissolved. The process reduced the foam viscosity and made it slightly malleable. The foam as in previous examples was shared into glass dishes, allowed to cool to room temperature, then stored in the fridge for 3 h before freezing overnight and freeze drying. The foam produced was hard, rather dense and more difficult to compress.

### Example 12: Psyllium Foam Containing Gelatin (Type B)

The foam preparation was as described in example 11 except for the use of gelatin obtained from bovine skin (Type B gel strength 225 g Bloom). This was to demonstrate the effect; if any, of the source of gelatin on the foam properties. The foam obtained was even harder than in example 11 and more difficult to press down.

### Example 13: Psyllium Foam Containing Gelatin (Type B), Tween 20 and Hydrogen Peroxide

This example was investigated in an attempt to produce psyllium-gelatin foams with improved absorbency and softer texture than hitherto. The two polymers (psyllium husk 7.5 g 1.5% w/w and gelatin 4.1 g 0.82% w/w) were mixed well together in powder form, then gradually added to RO water being stirred slowly in blender. Mixing continued at this low speed for 10 min before the addition of hydrogen peroxide (10 g 2% w/w) and Tween 20 (2.5 g 0.5% w/w) followed by further mixing but at the maximum speed for 35 min until the temperature was about 70° C. The solution was transferred into glass dishes as in previous examples, allowed to cool in a fridge overnight then stored in a freezer for 48 h before freezing drying over 40 h. Foams produces were very white, soft and flexible. The gels formed were almost integral even after compression with 5 kg weight except at 30 min where the integrity of the foam especially after compression was noticed to have reduced as the gelled foam became difficult to handle.

### Example 14: Psyllium Foam Containing Carboxymethyl Cellulose (CMC)

Two types of CMC, both obtained from Dow Wolff Cellulosics, GmbH were used to produce the foams. The first was a low viscosity, 110-160 cP for 1% solution CMC (WALOCEL CRT 100 GA) with a degree of substitution (DS) of 0.82-0.95%. The foam preparation involved weighing Frøskaller Psyllium (1.5% w/w; 7.5 g) and mixing it with CMC (0.8% w/w; 4 g) and adding the mixed powder gently to 488.5 g RO water stirred in a Philips blender initially at medium speed for 3 min and then at high speed for 7 minutes. The foam prepared appeared ‘stringy’ with big bubbles. It was transferred into small dishes, allowed to cool and stored in a freezer overnight before freeze drying.

The experiment was then repeated using the second type of CMC (WALOCEL CRT 10,000 GA) with higher viscosity CMC (1,370-1,500 cP for 1% solution CMC but with a similar degree of substitution (DS) of 0.90-0.95% and the foam mixed at viable speeds (2 min at minimum speed, 15 min at medium and 3 min maximum).

### Example 15: Psyllium Foam Containing High M Alginate

Alginate is a linear polymer consisting of two monomers, mannuronic acid (M) and guluronic acid (G). The ratio of these two monomers (the so called M:G ratio) and the proportion of the blocks of MM, MG and GG segments in the polymer vary from one alginate source to another and determine to a large extent the absorbent properties of the alginate. It is known that high M alginates produce softer and more absorbent gels. In this example, the foam prepared consists of psyllium (1% w/w, 5 g); high M alginate Manucol DH supplied by FMG (0.5% w/w, 2.5 g); lecithin (0.08% w/w, 0.4 g); propylene glycol (2% w/w, 10 g) and RO water (96.42% w/w, 482.1 g). To prepare the foam, the lecithin and propylene glycol were weighed and mixed into water in a blender and stirred for 5 min at high speed. Meanwhile, the psyllium and alginate were weighed, mixed together and then slowly transferred into the blender at minimum speed. After addition, the speed was increased and mixture stirred for further 5 min before sharing into glass dishes and allowing to cool to ambient temperature. The foam was stored in a freezer overnight before freeze drying over 48 h. Foams produced were soft and flexible.

### Example 16: Psyllium Foam Containing Alginate, Lecithin, CMC and Salts

In this example, combinations of various alginates (high G/high M grades), CMC, salts (calcium chloride dihydrate and ferric sulphate hydrate), lecithin and propylene glycol were used to produce the foam. The 4% foam solution was prepared by weighing and mixing together the following:
12 g Psyllium powder (obtained from Atlas Industries, India);
3 g CMC (WALOCEL CRT 1000 GA, Dow Wolff Cellulosics GmbH);
3 g High M alginate (Manucol DH, FMC BioPolymer)
1 g High G alginate (Protanal LF 10/60FT, FMC BioPolymer)
0.4 g Lecithin (L-alpha-lecithin, granular from soybean oil, Acros organics)
0.2 g calcium chloride dihydrate (>99% ACS grade CaCl₂.2H₂O; Alfar Aesar)
0.4 g Iron (III) sulphate hydrate (97% Fe₂(SO₄)₃.xH₂O, Sigma-Aldrich)

The mixture was then slowly added to 456 g RO water that was being stirred vigorously in a blender. The mixture was stirred continuously until all the solids were completely and homogeneously dissolved (30 min). This was then followed by a gradual addition of 24 g propylene glycol and further mixing until a very thick and consistent gel was obtained (10 min). The foam was then poured into glass dishes (each dish 10 cm dia./depth ˜1.5 cm and contained between 60-80 g foam). The dishes were placed in a fridge for 2 hours before storing in a freezer overnight and freeze drying for 50 hours. The foams produced were 6-8 mm thick, dense, flexible, soft but with rough surfaces and tough when pulled apart.

### Example 17: Psyllium Foam Containing Silk

2 g of regenerated silk produced ‘in house’ from Bombyx mori silk obtained from Thailand were added to 40% w/w (20 g) aqueous solution of lithium bromide in a flask, heated to boil and refluxed at the boiling temperature (80-90° C.) until all the silk dissolved (1 h) to produce solution 1. Meanwhile, 7.5 g of psyllium were weighed, added to 480 g RO water containing 2.5 g Tween 20 and mixed until dissolved (6 min) to produce solution 2. The two solutions were then mixed together for 10 minutes at a very high speed in a blender to produce a thick foaming solution. The solution was poured into glass dishes at weights 60-90 g, cooled in a fridge before storing in a freezer overnight. The solid foam was then freeze dried for 60 hours. Foams (2-3 mm thick) produced with 1.6% w/w LiBr were very rough, brittle and hard when removed from the freeze dryer. However, after storing in a conditioning chamber (65% RH/20° C.) for just about 3 minutes or in the laboratory atmosphere (24% RH/21° C.) for 15 min, the foams became flexible, soft to handle and tough when stretched.

### Example 18a: Psyllium Foam Prepared by Drying in the Oven

This example was investigated to assess the effect of drying the foam in an oven instead of freeze drying. The psyllium powder (15 g, 3% w/w obtained from Atlas Industries, India) was mixed into 450 g of RO water to a smooth paste (2 minutes). Propylene glycol (25 g, 5% w/w) and Tween 20 (6 g, 1% w/w) were mixed together and then gradually added to the mixing psyllium paste. This was followed immediately by the addition of 3 g (1% w/w) calcium chloride dihydrate. The mixture was stirred until the foam formed could be lifted wholly out of the jar into a tray, spread out thinly (1 cm thick) and left in an oven initially at 65° C. for 7 h, then at reduced temperature (40° C.) for 2 days. The resulting foam was flexible especially when thin, comfortable to touch, stretchy and sticky.

### Example 18b: Psyllium Foam Prepared by Drying in the Oven

In this example, the psyllium husk (20 g, 2% w/w) was soaked in water (40° C.) for 30 minutes, then mixed for 5 minutes before a gradual addition of glycerol (10 g 1% w/w), hydrogen peroxide (50 g 5% w/w) and mixing at high speed for further 5 minutes. The foam was then transferred into a glass tray, spread to about 20 mm thick and dried in an oven at 70° C. for 30 h. The foam created was tough with a dried skin on the external surface.

### Foam Liquid Absorption and Retention Characteristics

The absorbency and retention properties of the psyllium foams of examples 1 to 18b were assessed by immersing the examples in the following:
1. Solution A (mixed CaCl₂).2H₂O and NaCl aq. solution containing 142 mmol/litre Na⁺ ions, 8.298 g NaCl and 2.5 mmol/litre of Ca²⁺ ions, 0.368 g CaCl₂.2H₂O)
2. Normal saline (0.9% or 153.8 mmol or 9 g of NaCl/litre)

### a) Absorbency

Absorbency was determined with reference to BS EN standard (13726-1:2002). In summary, the foam sample (1×1 cm, weight W1) was fully immersed in 30 g saline (37° C.) or solution A (37° C.) contained in ‘200 ml plastic water cup’ and allowed to stand for 1 or 3 or 30 min in oven at 37° C. The sample was removed, then allowed to drain for seconds and weighed (W2). The absorbent capacity (g/g) was calculated as a ratio of the wet weight (W2−W1) of the foam to the dry weight (W1) at ambient temperature

Absorbency (g/g)=(W2−W1)/W1

### b) Liquid Retention

The wet foam (W2) was then placed on a perforated metal plate. A Perspex plate was laid over the foam to ensure even pressure distribution and a weight 5 kg (equivalent to 40 mm Hg as commonly applied with a high compression bandage therapy) was applied to the foam for 10 seconds and removed. Any unbound or excess liquid expelled was allowed to drain into a tray; then the pressured foam was reweighed (W3) and retention (g/g) calculated as follows:

Retention (g/g)=(W3−W1)/W1

Retention (%)=(W3−W1)×100/(W2−W1)

### Irradiation Treatment (Gamma Sterilisation)

As with most medical devices, gamma radiation sterilisation technique was used to sterilise some of the foams to determine effects on stability as the technique can sometimes damage polymer materials resulting in chain scission and reduced integrity or crosslinking or discoloration.

Gamma sterilisation (Synergy Health, UK) was carried out using a dose of 25 kGy. Irradiation testing revealed that irradiation has no effect on the absorbent properties of psyllium foam, although on close examination there appeared to be a general decrease in fluid retention from an average of 82% to 70% in solution A and from 85% to 77% in saline indicating that the effect was slightly more pronounced in solution A. However, the gels formed in these fluids remained integral for both irradiated and non-irradiated samples.

The absorbency and retention results for each of the examples 1 to 18b are detailed below.

TABLE 2
Absorbency and retention of psyllium foam prepared according to example 1 (non-irradiated sample)

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |
| | SOLUTION A | | | | | | | |
| P-160330 | 0.0168 | 1 | 0.5501 | 0.4467 | 0.53 | 31.74 | 0.43 | 25.59 |
| | 0.0177 | 3 | 0.6615 | 0.5763 | 0.64 | 36.37 | 0.56 | 31.56 |
| | 0.0168 | 30 | 0.8911 | 0.7191 | 0.87 | 52.04 | 0.70 | 41.80 |
| | SALINE SOLUTION | | | | | | | |
| | 0.0161 | 1 | 0.5338 | 0.4291 | 0.5177 | 32.15 | 0.43 | 25.65 |
| | 0.0151 | 3 | 0.5153 | 0.4398 | 0.5002 | 33.12 | 0.56 | 28.12 |
| | 0.0164 | 30 | 0.8493 | 0.7736 | 0.8329 | 50.78 | 0.70 | 46.17 |

TABLE 3
Absorbency and retention of psyllium foam prepared according to example 1 (irradiated sample)

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |
| | SOLUTION A | | | | | | | |
| P-160330 | 0.0234 | 1 | 0.8118 | 0.4982 | 0.7884 | 33.69 | 0.4748 | 20.29 |
| | 0.0264 | 3 | 0.9503 | 0.6260 | 0.9239 | 35.00 | 0.5996 | 22.71 |
| | 0.0241 | 30 | 1.3545 | 1.1476 | 1.3304 | 55.20 | 1.1235 | 46.62 |
| | SALINE SOLUTION | | | | | | | |
| | 0.0211 | 1 | 0.7145 | 0.5625 | 0.6934 | 32.86 | 0.5414 | 25.66 |
| | 0.0219 | 3 | 0.7550 | 0.5436 | 0.7331 | 33.47 | 0.5217 | 23.82 |
| | 0.0217 | 30 | 1.2387 | 1.0342 | 1.2170 | 56.08 | 1.0125 | 46.66 |

Both the non-irradiated and irradiated foams were highly absorbent and exhibited excellent retention. The results in the tables 2 and 3 appear to show that irradiation has no effect on the absorbent properties of psyllium foam, although on close examination there appeared to be a general decrease in fluid retention from an average of 82% to 70% in solution A and from 85% to 77% in saline indicating that the effect was slightly more pronounced in solution A. However, the gels formed in these fluids remained integral for both irradiated and non-irradiated samples.

TABLE 4
Absorbent properties of psyllium foam of example 2 containing hydrogen peroxide and Tween 20

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |

SOLUTION A

| | | | | | | | | |
|---|---|---|---|---|---|---|---|---|
| P-160920/ | 0.0626 | 1 | 1.0658 | 0.8834 | 1.0032 | 16.03 | 0.8208 | 13.11 |
| H₂O₂/ | 0.0571 | 3 | 0.8885 | 0.7608 | 0.8314 | 14.56 | 0.7037 | 12.32 |
| Teen 20 | 0.0706 | 30 | 1.2501 | 1.1859 | 1.1795 | 16.71 | 1.1153 | 15.80 |

SALINE SOLUTION

| | | | | | | | | |
|---|---|---|---|---|---|---|---|---|
| P-160920/ | 0.0388 | 1 | 0.7318 | 0.6093 | 0.6930 | 17.86 | 0.5705 | 14.70 |
| H₂O₂/ | 0.0337 | 3 | 0.5890 | 0.4963 | 0.5553 | 16.48 | 0.4626 | 13.73 |
| Teen 20 | 0.0349 | 30 | 1.2738 | 1.1148 | 1.2389 | 35.47 | 1.0799 | 30.94 |

TABLE 5
Absorbent properties of psyllium foam of example 3 prepared from unfiltered psyllium
solution and containing hydrogen peroxide and Tween 20

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |

SOLUTION A

| | | | | | | | | |
|---|---|---|---|---|---|---|---|---|
| P-160928/ | 0.0395 | 1 | 1.0336 | 0.8218 | 0.9941 | 25.17 | 0.7823 | 19.81 |
| H₂O₂/ | 0.0410 | 3 | 1.2618 | 0.9640 | 1.2208 | 29.78 | 0.9230 | 22.51 |
| Teen 20 | 0.0372 | 30 | 1.4017 | 1.1268 | 1.3645 | 36.68 | 1.0896 | 29.29 |

SALINE SOLUTION

| | | | | | | | | |
|---|---|---|---|---|---|---|---|---|
| P-160928/ | 0.0346 | 1 | 1.2052 | 0.8840 | 1.1706 | 33.83 | 0.8494 | 24.55 |
| H₂O₂/ | 0.0348 | 3 | 1.2870 | 1.0201 | 1.2522 | 35.98 | 0.9858 | 28.31 |
| Teen 20 | 0.0301 | 30 | 1.2630 | 1.0782 | 1.2329 | 40.96 | 1.0481 | 34.82 |

In solution A and saline, the foams were very integral even after 30 mins and absorbency and retention increased substantially and continuously with storage time, although as perhaps expected the softer foam was more absorbent but only slightly.

TABLE 6
Effect of high psyllium concentration on foam absorbent properties of example 4

| | | | Weight | | | | | |
|---|---|---|---|---|---|---|---|---|
| | Dry | Im- | after liquid | Weight | Absorbency | | Retention | |
| | weight | mersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |

SOLUTION A

| | | | | | | | | |
|---|---|---|---|---|---|---|---|---|
| P-160923/ | 0.0460 | 1 | 0.7958 | 0.6835 | 0.7498 | 16.30 | 0.6375 | 13.86 |
| H₂O₂/Tween20 | 0.0466 | 3 | 1.0484 | 0.8639 | 1.0018 | 21.50 | 0.8173 | 17.54 |
| (5% w/w | 0.0394 | 30 | 1.0233 | 0.9165 | 0.9830 | 24.97 | 0.9650 | 22.26 |
| psyllium) | | | | | | | | |

SALINE SOLUTION

| | | | | | | | | |
|---|---|---|---|---|---|---|---|---|
| P-160923/ | 0.0630 | 1 | 0.7360 | 0.6774 | 0.6730 | 10.68 | 0.6144 | 9.75 |
| H₂O₂/Tween20 | 0.0603 | 3 | 1.0082 | 0.9093 | 0.9479 | 15.58 | 0.8490 | 13.96 |
| (5% w/w | 0.0538 | 30 | 1.2851 | 1.1840 | 1.2313 | 22.89 | 1.1302 | 21.01 |
| psyllium) | | | | | | | | |

TABLE 7
Effect of addition of glycerol and lecithin on psyllium foam of example 5 (non-irradiated)

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |

SOLUTION A

| P-160725/ | 0.1149 | 1 | 0.2760 | 0.2329 | 0.1611 | 1.40 | 0.1180 | 1.03 |
|---|---|---|---|---|---|---|---|---|
| H₂O₂/ | 0.1533 | 3 | 0.4169 | 0.3731 | 1.0018 | 1.72 | 0.2198 | 1.43 |
| LE/Gly | 0.1116 | 30 | 0.7059 | 0.6608 | 0.9627 | 5.33 | 0.5492 | 4.92 |
| | SALINE SOLUTION | | | | | | | |
| | 0.1881 | 1 | 0.4124 | 0.3752 | 0.2243 | 1.19 | 0.1871 | 0.99 |
| | 0.1758 | 3 | 0.4555 | 0.4182 | 0.2797 | 1.59 | 0.2424 | 1.38 |
| | 0.1653 | 30 | 0.6853 | 0.6426 | 0.5200 | 3.15 | 0.4773 | 2.89 |

The results of table 7 indicate a foam with very much reduced absorbency and retention when compared with previous examples. The low absorbency may be attributed to the high glycerol content in the foam.

TABLE 8
Effect of addition of glycerol and lecithin on psyllium foam of example 5 (irradiated)

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |
| | SOLUTION A | | | | | | | |
| P-160725/ | 0.2414 | 1 | 0.4376 | 0.4193 | 0.1962 | 0.81 | 0.1779 | 0.74 |
| H₂O₂/ | 0.1841 | 3 | 0.4242 | 0.3988 | 0.2401 | 1.30 | 0.2147 | 1.17 |
| LE/Gly | 0.1867 | 30 | 0.6690 | 0.6210 | 0.4823 | 2.58 | 0.4343 | 2.33 |
| | SALINE SOLUTION | | | | | | | |
| 0.1763 | | 1 | 0.3676 | 0.3364 | 0.1913 | 1.09 | 0.1601 | 0.91 |
| 0.1775 | | 3 | 0.3762 | 0.3514 | 0.1987 | 1.12 | 0.1739 | 0.98 |
| 0.1600 | | 30 | 0.5510 | 0.5062 | 0.3910 | 2.44 | 0.3462 | 2.16 |

From table 8 it appears irradiation leads to a small reduction in the foam fluid properties tested.

TABLE 9
Effect of addition of lecithin, propylene glycol, hydrogen peroxide and calcium salt
on absorbency of a foam of example 6

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |

SOLUTION A

| | | | | | | | | |
|---|---|---|---|---|---|---|---|---|
| P-160930/ | 0.0748 | 1 | 0.5972 | 0.5231 | 0.5224 | 6.98 | 0.4483 | 5.99 |
| H₂O₂/ | 0.0844 | 3 | 0.7465 | 0.6829 | 0.6621 | 7.84 | 0.5985 | 7.09 |
| LE/PG/Ca | 0.0770 | 30 | 1.0089 | 0.9466 | 0.9319 | 12.10 | 0.8696 | 11.29 |

SALINE SOLUTION

| | | | | | | | | |
|---|---|---|---|---|---|---|---|---|
| P-160930/ | 0.0685 | 1 | 0.5731 | 0.5320 | 0.5046 | 7.37 | 0.4635 | 6.77 |
| H₂O₂/ | 0.0751 | 3 | 0.6592 | 0.6454 | 0.5841 | 7.78 | 0.5703 | 7.59 |
| LE/PG/Ca | 0.0723 | 30 | 0.8028 | 0.7229 | 0.7305 | 10.10 | 0.6506 | 9.00 |

From table 9, absorbency and retention indicate a very light foam with moderate absorbency/retention without loss in integrity in solution.

TABLE 10
Absorbency of psyllium containing glycerol, propylene glycol and hydrogen
peroxide according to example 7

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |

SOLUTION A

| | | | | | | | | |
|---|---|---|---|---|---|---|---|---|
| P-160310/ | 0.0479 | 1 | 0.5498 | 0.4661 | 0.5019 | 10.48 | 0.4182 | 8.73 |
| PG/Gly/ | 0.0413 | 3 | 0.5869 | 0.5161 | 0.5456 | 13.21 | 0.4748 | 11.50 |
| H₂O₂ | 0.0440 | 30 | 0.8220 | 0.6817 | 0.7780 | 17.68 | 0.6377 | 14.49 |

SALINE SOLUTION

| | | | | | | | | |
|---|---|---|---|---|---|---|---|---|
| P-160310/ | 0.0484 | 1 | 0.5911 | 0.5218 | 0.5427 | 11.21 | 0.4734 | 9.78 |
| PG/Gly/ | 0.0432 | 3 | 0.7088 | 0.5860 | 0.6656 | 15.41 | 0.5428 | 12.56 |
| H₂O₂ | 0.0402 | 30 | 0.9338 | 0.8318 | 0.8936 | 22.23 | 0.7916 | 19.69 |

From table 10, absorbency and retention were moderate and integrity was good in the solutions irrespective of immersion time.

TABLE 11
Absorbency of psyllium foam according to example 8 containing chitosan (non-irradiated)

| | | | Weight | | | | | |
|---|---|---|---|---|---|---|---|---|
| | Dry | | after liquid | Weight | Absorbency | | Retention | |
| | weight | Immersion | absorption/ | after com- | | (W2- | | (W3- |
| Batch | W1 | time | draining | pression | W2-W1 | W1)/W1 | W3-W1 | W1)/W1 |
| number | (g) | (min) | W2 (g) | W3 (g) | (g) | (g/g) | (g) | (g/g) |
| | SOLUTION A | | | | | | | |
| CELOX | 0.0324 | 1 | 0.2406 | 0.2014 | 0.21 | 6 .43 | 0.17 | 5.22 |
| P-160708- | 0.0330 | 1 | 0.8071 | 0.5171 | 0.74741 | 23.46 | 0.4833 | 14.67 |
| CS | 0.0352 | 3 | 0.9067 | 0.5713 | 0.8715 | 24.76 | 0.5361 | 15.23 |
| | 0.0363 | 30 | 1.1032 | 0.8018 | 1.0669 | 29.39 | 0.7655 | 21.09 |
| | SALINE SOLUTION | | | | | | | |
| | 0.0331 | 1 | 0.6873 | 0.3542 | 0.6542 | 19.76 | 0.3211 | 9.70 |
| | 0.0362 | 3 | 0.9757 | 0.4849 | 0.9395 | 25.95 | 0.4489 | 12.37 |
| | 0.0318 | 30 | 1.1040 | 0.6689 | 1.0722 | 33.71 | 0.6371 | 20.03 |

TABLE 12
Absorbency of psyllium foam according to example 8 containing chitosan (irradiated)

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |
| | SOLUTION A | | | | | | | |
| P-160708-CS | 0.0339 | 1 | 0.7389 | 0.5518 | 0.7050 | 20.80 | 0.5179 | 15.28 |
| | 0.0385 | 3 | 0.9135 | 0.7096 | 0.8750 | 22.73 | 0.6711 | 17.43 |
| | 0.0352 | 30 | 1.0552 | 0.8079 | 1.0200 | 28.98 | 0.7727 | 21.95 |
| | SALINE SOLUTION | | | | | | | |
| | 0.0406 | 1 | 0.9181 | 0.6972 | 0.8775 | 21.61 | 0.6566 | 16.17 |
| | 0.0401 | 3 | 1.0110 | 0.6276 | 0.9709 | 24.21 | 0.5875 | 14.65 |
| | 0.0402 | 30 | 1.1420 | 0.7977 | 1.1018 | 27.41 | 0.7575 | 18.84 |

The foam of example 8 absorbed well within 1 minute in either solution A. In fact, in comparison with commercial product, CELOX (Medtrade Products Ltd, Crewe, UK), it was about 3 times more absorbent within 1 minute of immersion in the liquid although retention was lower for psyllium-chitosan foams. Irradiation (Table 12) has not affected the absorbency profile to any significant extent, although a very slight decrease in absorbency and increase in retention was noticed

TABLE 13
Absorbency of psyllium foam according to example 9 containing pectin (non-irradiated)

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |
| | SOLUTION A | | | | | | | |
| P-161020-PE/ | 0.0502 | 1 | 1.3278 | 1.1707 | 1.2776 | 25.45 | 1.1205 | 22.32 |
| H₂O₂/Tween 20 | 0.0548 | 3 | 1.8017 | 1.5148 | 1.7469 | 31.88 | 1.4600 | 26.64 |
| | 0.0549 | 30 | 2.6073 | 2.1253 | 2.5524 | 46.49 | 2.0704 | 37.71 |
| | SALINE SOLUTION | | | | | | | |
| | 0.0430 | 1 | 1.3430 | 1.0904 | 1.3000 | 30.23 | 1.0474 | 24.36 |
| | 0.0300 | 3 | 1.1708 | 0.9026 | 1.408 | 38.02 | 0.8726 | 29.09 |
| | 0.0399 | 30 | 1.7989 | 1.2370 | 1.7590 | 44.08 | 1.1971 | 30.00 |

TABLE 14
Absorbency of psyllium foam according to example 9 containing pectin (irradiated)

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |
| | SOLUTION A | | | | | | | |
| P-161020-PE/ | 0.0451 | 1 | 1.1128 | 0.8112 | 1.0677 | 23.67 | 0.7661 | 16.99 |
| H₂O₂/Tween | 0.0523 | 3 | 1.3285 | 1.0561 | 1.2762 | 24.40 | 1.0038 | 19.19 |
| 20 | 0.0497 | 30 | 1.6270 | 1.3745 | 1.5773 | 31.74 | 1.3248 | 26.65 |
| | SALINE SOLUTION | | | | | | | |
| | 0.0440 | 1 | 1.1112 | 0.8616 | 1.0672 | 24.25 | 0.8176 | 18.58 |
| | 0.0414 | 3 | 1.1180 | 0.7906 | 1.0766 | 26.00 | 0.7492 | 18.10 |
| | 0.0344 | 30 | 1.2509 | 1.0091 | 1.2165 | 35.36 | 0.9747 | 28.33 |

Absorbency of a foam of example 9 in solution A or in saline was immediate and substantial but with loss of integrity and weak gel with immersion time in either liquid. Foam irradiated (Table 14) appeared to have slightly lower absorbency.

TABLE 15
Absorbency of psyllium foam according to example 10 containing pectin, a foaming agent, hydrogen peroxide and calcium chloride

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |

SOLUTION A

| | | | | | | | | |
|---|---|---|---|---|---|---|---|---|
| P-161021-PE/ | 0.0677 | 1 | 0.8781 | 0.7548 | 0.8100 | 11.96 | 0.6871 | 10.15 |
| H₂O₂/Tween | 0.0547 | 3 | 1.1773 | 0.7880 | 1.1126 | 20.52 | 0.7333 | 13.41 |
| 20 /Ca | 0.0524 | 30 | 1.0640 | 0.8361 | 1.0116 | 19.30 | 0.7837 | 14.96 |

SALINE SOLUTION

| | | | | | | | | |
|---|---|---|---|---|---|---|---|---|
| P-161021-PE/ | 0.0538 | 1 | 0.7758 | 0.6809 | 0.7220 | 13.42 | 0.6271 | 11.66 |
| H₂O₂/Tween | 0.0596 | 3 | 1.0444 | 0.9286 | 0.9848 | 16.51 | 0.8690 | 14.58 |
| 20 /Ca | 0.0459 | 30 | 0.8231 | 0.6184 | 0.7772 | 16.93 | 0.5725 | 12.46 |

For example 10, addition of salt and slight surface hardness appeared to reduce foam absorbency in comparison with example 9. Integrity improved only slightly.

TABLE 16
Absorbency of psyllium foam of example 11 containing porcine gelatin

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |

SOLUTION A

| | | | | | | | | |
|---|---|---|---|---|---|---|---|---|
| P-160901-GeP | 0.0661 | 1 | 0.5965 | 0.5191 | 0.5304 | 8.02 | 0.4530 | 6.85 |
| | 0.0757 | 3 | 0.8210 | 0.7048 | 0.7553 | 9.85 | 0.6321 | 8.35 |
| | 0.0787 | 30 | 0.9565 | 0.8324 | 0.8778 | 11.15 | 0.7537 | 9.58 |

SALINE SOLUTION

| | | | | | | | | |
|---|---|---|---|---|---|---|---|---|
| P-160901-GeP | 0.0610 | 1 | 0.8248 | 0.6845 | 0.7638 | 12.52 | 0.6235 | 10.22 |
| | 0.0589 | 3 | 1.1113 | 0.9982 | 1.0521 | 17.86 | 0.9393 | 15.95 |
| | 0.0635 | 30 | 0.8458 | 0.7121 | 0.7823 | 12.32 | 0.6486 | 10.21 |

For a foam of example 11, absorbency was on the low side even after immersing in solution A for 30 mins but integrity was almost good throughout except after 30 min in saline. The rather low integrity in saline after 30 mins led to some loss of gelled foam during the process.

TABLE 17
Absorbency of psyllium foam according to example 12 containing bovine gelatin

| | | | Weight after | Weight | | | | |
|---|---|---|---|---|---|---|---|---|
| | | | liquid | after | Absorbency | | Retention | |
| | Dry weight | Immersion | absorption/ | compres- | | (W2 − | | (W3 − W1)/ |
| | W1 | time | draining W2 | sion W3 | W2 − W1 | W1)/W1 | W3 − W1 | W1 |
| Batch number | (g) | (min) | (g) | (g) | (g) | (g/g) | (g) | (g/g) |

SOLUTION A

| | | | | | | | | |
|---|---|---|---|---|---|---|---|---|
| P-160902- | 0.0636 | 1 | 0.6126 | 0.4956 | 0.5490 | 8.63 | 0.4320 | 6.80 |
| GeB | 0.0776 | 3 | 0.9809 | 0.7145 | 0.9033 | 11.64 | 0.6369 | 8.20 |
| | 0.0724 | 30 | 0.9190 | 0.7191 | 0.8466 | 11.69 | 0.6467 | 8.94 |

SALINE SOLUTION

| | | | | | | | | |
|---|---|---|---|---|---|---|---|---|
| P-160902- | 0.0756 | 1 | 0.7414 | 0.6144 | 0.6658 | 8.81 | 0.5388 | 7.13 |
| GeB | 0.0909 | 3 | 0.8371 | 0.7154 | 0.7462 | 8.21 | 0.6245 | 6.87 |
| | 0.0696 | 30 | 0.8979 | 0.7470 | 0.8283 | 11.90 | 0.6774 | 9.73 |

SOLUTION A

| | | | | | | | | |
|---|---|---|---|---|---|---|---|---|
| SPONGOSTAN | 0.0179 | 1 | 0.6337 | 0.2290 | 0.62 | 34.4 | 0.21 | 11.79 |
| | 0.0155 | 3 | 0.3541 | 0.2345 | 0.34 | 21.85 | 0.22 | 14.33 |
| | 0.0193 | 30 | 0.6653 | 0.2656 | 0.65 | 33.47 | 0.25 | 12.76 |

For foams of example 12, absorbency was similar to that obtained for porcine gelatin but surprisingly the foam was less integral especially in solution A. The properties obtained were lower than those observed for the commercial gelatin foam, SPONGOSTAN (Ethicon, US), though fluid retention in Spongostan foam was rather low (average retention for the 1, 3 and 30 minutes tests in solution A was about 45% compared to 75% in the present invention for type B gelatin). In general though, it appears that the psyllium/gelatin foams irrespective of the source of gelatin have low absorbency when compared with some of the 100% psyllium foams or psyllium/pectin foams in example 1 and in examples 9 and 10 respectively.

TABLE 18
Absorbency of psyllium foam of example 13 containing bovine gelatin, foaming agent and hydrogen peroxide (non-irradiated)

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |

SOLUTION A

| | | | | | | | | |
|---|---|---|---|---|---|---|---|---|
| P-161205-GeB/ | 0.0448 | 1 | 0.7402 | 0.6223 | 0.6954 | 15.52 | 0.5775 | 12.88 |
| H₂O₂/Tween 20 | 0.0430 | 3 | 0.8075 | 0.6436 | 0.7645 | 17.78 | 0.6006 | 13.95 |
| | 0.0484 | 30 | 0.9731 | 0.8394 | 0.9247 | 19.11 | 0.7910 | 16.34 |

SALINE SOLUTION

| | | | | | | | | |
|---|---|---|---|---|---|---|---|---|
| P-161205-GeB/ | 0.0405 | 1 | 0.7546 | 0.6035 | 0.7141 | 17.63 | 0.5630 | 13.90 |
| H₂O₂/Tween 20 | 0.0517 | 3 | 0.9340 | 0.7729 | 0.8823 | 17.06 | 0.7212 | 13.95 |
| | 0.0457 | 30 | 0.8157 | 0.7088 | 0.7700 | 16.85 | 0.6631 | 14.51 |

TABLE 19
Absorbency of psyllium foam of example 13 containing bovine gelatin, foaming agent and hydrogen peroxide (irradiated)

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |
| | SOLUTION A | | | | | | | |
| P-161205-GeB/ | 0.0516 | 1 | 0.8529 | 0.5641 | 0.8013 | 15.53 | 0.5125 | 9.93 |
| H₂O₂/Tween | 0.0512 | 3 | 0.8885 | 0.6510 | 0.8373 | 16.35 | 0.5998 | 11.71 |
| 20 | 0.0414 | 30 | 0.8985 | 0.6612 | 0.8571 | 20.70 | 0.6198 | 14.97 |
| | SALINE SOLUTION | | | | | | | |
| | 0.0533 | 1 | 0.8210 | 0.5340 | 0.7677 | 14.40 | 0.4807 | 9.02 |
| | 0.0543 | 3 | 0.8752 | 0.5975 | 0.8209 | 15.12 | 0.5432 | 10.00 |
| | 0.0453 | 30 | 0.8644 | 0.7011 | 0.8191 | 18.08 | 0.6558 | 14.48 |

For the foams of example 13, irradiation appeared to have no significant effect on the foam absorbency and retention.

TABLE 20
Effect of CMC viscosity (molecular weight) on the absorbency of psyllium-CMC foams according to example 14

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |

SOLUTION A

| | | | | | | | | |
|---|---|---|---|---|---|---|---|---|
| P-161212-CMC | 0.0614 | 1 | 0.8345 | 0.7321 | 0.7731 | 12.59 | 0.6707 | 10.92 |
| Low viscosity CMC | 0.0614 | 3 | 0.9751 | 0.8328 | 0.9137 | 14.88 | 0.7714 | 12.56 |
| | 0.0631 | 30 | 0.9501 | 0.8498 | 0.8870 | 14.06 | 0.7867 | 12.47 |

SALINE SOLUTION

| | | | | | | | | |
|---|---|---|---|---|---|---|---|---|
| P-161212-CMC | 0.0620 | 1 | 0.9968 | 0.8086 | 0.9348 | 15.08 | 0.7466 | 12.04 |
| Low viscosity CMC | 0.0656 | 3 | 1.1958 | 1.0197 | 1.1302 | 17.23 | 0.9541 | 14.54 |
| | 0.0663 | 30 | 1.0948 | 0.9701 | 1.0285 | 15.51 | 0.9038 | 13.63 |

SOLUTION A

| | | | | | | | | |
|---|---|---|---|---|---|---|---|---|
| P-161212-CMC | 0.0474 | 1 | 0.9556 | 0.8321 | 0.9082 | 19.16 | 0.7847 | 16.55 |
| high viscosity CMC | 0.0355 | 3 | 0.9428 | 0.8499 | 0.9073 | 25.56 | 0.8144 | 22.94 |
| | 0.0476 | 30 | 1.2500 | 1.1647 | 1.2024 | 25.26 | 1.1171 | 23.47 |

SALINE SOLUTION

| | | | | | | | | |
|---|---|---|---|---|---|---|---|---|
| P-161212-CMC | 0.0349 | 1 | 0.9240 | 0.8166 | 0.8891 | 25.47 | 0.7817 | 22.40 |
| high viscosity CMC | 0.0357 | 3 | 0.8255 | 0.7264 | 0.7898 | 22.12 | 0.6907 | 19.35 |
| | 0.0593 | 30 | 1.5544 | 1.3797 | 1.4951 | 25.21 | 1.3204 | 22.27 |

For the foams of example 14, generally the results indicated a rapid absorbency within the first imin, after which not much increase was observed even after 30 min immersion. The results also showed improvement in absorbency with increasing CMC viscosity or molecular weight but the foam integrity decreased especially after 30 minutes. Fluid retention as a whole was high at about 85% for the two types of CMC.

TABLE 21
Absorbent properties of psyllium foam according to example 15 containing high M alginate, lecithin and propylene glycol

| | | | Weight after | Weight | | | | |
|---|---|---|---|---|---|---|---|---|
| | | | liquid | after | Absorbency | | Retention | |
| | Dry weight | Immersion | absorption/ | compres- | | (W2 − | | (W3 − W1)/ |
| | W1 | time | draining W2 | sion W3 | W2 − W1 | W1)/W1 | W3 − W1 | W1 |
| Batch number | (g) | (min) | (g) | (g) | (g) | (g/g) | (g) | (g/g) |

SOLUTION A

| | | | | | | | | |
|---|---|---|---|---|---|---|---|---|
| P-161214- | 0.0543 | 1 | 0.4909 | 0.4516 | 0.436 | 8.03 | 0.0397 | 7.31 |
| HM/LE/PG | 0.0501 | 3 | 0.6523 | 0.5296 | 0.602 | 12.02 | 0.479 | 9.56 |
| | 0.0512 | 30 | 0.7971 | 0.7047 | 0.745 | 14.55 | 0.653 | 12.75 |

SALINE SOLUTION

| | | | | | | | | |
|---|---|---|---|---|---|---|---|---|
| P-161214- | 0.0689 | 1 | 0.4851 | 0.3788 | 0.416 | 6.04 | 0.309 | 4.48 |
| HM/LE/PG | 0.0523 | 3 | 0.5391 | 0.4873 | 0.486 | 9.29 | 0.435 | 8.32 |
| | 0.0555 | 30 | 0.7673 | 0.6486 | 0.711 | 12.81 | 0.593 | 10.68 |

Based on the results of table 21 (example 15) absorbency in solution A and saline increased with time; although initial absorbencies in both fluids were rather low. However, percentage retentions were high at about 90% in both solutions but decreased with immersion time.

TABLE 22
Absorbent properties of psyllium foam according to example 16 containing alginate,
CMC, lecithin, propylene glycol, calcium and iron salts

| | |
|---|---|
| Weight after | Weight |

| | | | liquid | after | Absorbency | | Retention | |
|---|---|---|---|---|---|---|---|---|
| | Dry weight | Immersion | absorption/ | compres- | | (W2 − | | (W3 − W1)/ |
| | W1 | time | draining W2 | sion W3 | W2 − W1 | W1)/W1 | W3 − W1 | W1 |
| Batch number | (g) | (min) | (g) | (g) | (g) | (g/g) | (g) | (g/g) |

SOLUTION A

| P-160804- | 0.2015 | 1 | 0.5586 | 0.5263 | 0.3571 | 1.77 | 0.3248 | 1.61 |
|---|---|---|---|---|---|---|---|---|
| Hm/HG/CMC/PG/ | 0.2012 | 3 | 0.6034 | 0.5834 | 0.4022 | 2.00 | 0.3822 | 1.90 |
| LE/Ca/Fe | 0.1920 | 30 | 0.8915 | 0.8259 | 0.6995 | 3.64 | 0.6339 | 3.30 |
| | SALINE SOLUTION | | | | | | | |
| | 0.2105 | 1 | 0.6184 | 0.5384 | 0.4079 | 1.94 | 0.3279 | 1.56 |
| | 0.2045 | 3 | 0.6899 | 0.6380 | 0.4854 | 2.37 | 0.4335 | 2.12 |
| | 0.12201 | 30 | 1.0610 | 0.9633 | 0.8409 | 3.82 | 0.7432 | 3.38 |

TABLE 23
Effect of freeze drying a partially frozen psyllium foam of example 16

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/ |
| Batch number | (g) | (min) | (g) | (g) | (g) | (g/g) | (g) | W1(g/g) |
| | SOLUTION A | | | | | | | |
| [symbol] -160804- | 0.0590 | 1 | 0.4703 | 0.4240 | 0.4113 | 6.97 | 0.3650 | 6.19 |
| Hm/HG/CMC/PG/LE/Ca/Fe | 0.0450 | 3 | 0.4068 | 0.3557 | 0.3618 | 8.04 | 0.3107 | 6.90 |
| Partially frozen | 0.0683 | 30 | 0.7583 | 0.6875 | 0.6900 | 10.10 | 0.6192 | 9.07 |
| and freeze dried | SALINE SOLUTION | | | | | | | |
| | 0.1001 | 1 | 0.6320 | 0.5760 | 0.5319 | 5.3 | 0.4759 | 4.75 |
| | 0.0830 | 3 | 0.6523 | 0.5849 | 0.5693 | 6.86 | 0.5019 | 6.05 |
| | 0.1138 | 30 | 1.5924 | 1.3419 | 1.4786 | 12.99 | 1.2281 | 10.79 |

[symbol]

indicates data missing or illegible when filed

The absorbency of the foams of example 16 were very low but retention over 90% in both solution A and saline even after storing in the liquids for 30 minutes. An attempt was made to improve the absorbency and to observe the effect of the state of foam on freeze drying. Following the same procedure as above, a small amount of foam was prepared and briefly (30 minutes) left in the freezer and then freeze dried for 48 h. As expected, the volume of the foam increased initially but stabilized at the increased volume throughout the drying process. Foam produced was more porous, but internal structure was rather inconsistent and too porous. However, the foam (3-6 mm thick) was lighter, had better absorbency than those obtained from samples frozen to solid but lacked integrity especially after compression.

TABLE 24
Absorbent properties of psyllium-silk foam (average of several test results) according to example 17

| | | | Weight after | Weight | | | | |
|---|---|---|---|---|---|---|---|---|
| | | | liquid | after | Absorbency | | | |
| | Dry weight | Immersion | absorption/ | compres- | | (W2 − | Retention | |
| | W1 | time | draining W2 | sion W3 | W2 − W1 | W1)/W1 | W3 − W1 | (W3 − W1)/W1 |
| Batch number | (g) | (min) | (g) | (g) | (g) | (g/g) | (g) | (g/g) |
| | SOLUTION A | | | | | | | |
| P-170118- | 0.0763 | 1 | 0.4971 | 0.4314 | 0.4208 | 5.86 | 0.3551 | 4.94 |
| SF/LiBr/Tween 20 | 0.0772 | 3 | 0.5603 | 0.4835 | 0.4831 | 6.44 | 0.4063 | 5.43 |
| Psyllium-silk foam | 0.0758 | 30 | 0.8587 | 0.7426 | 0.7829 | 11.05 | 0.6668 | 9.38 |
| | SALINE SOLUTION | | | | | | | |
| | 0.0672 | 1 | 0.4924 | 0.4203 | 0.4252 | 6.66 | 0.3531 | 5.54 |
| | 0.1959 | 3 | 0.4527 | 0.3918 | 0.3874 | 6.09 | 0.3265 | 5.13 |
| | 0.0714 | 30 | 0.8266 | 0.7248 | 0.7552 | 10.04 | 0.6533 | 8.67 |

Regarding the foams of example 17 (based on the results of table 24), the foams swelled fairly rapidly in fluids to give integral gels but absorbency was generally low and only increased moderately even after 30 min in the fluids. As expected, attempts with higher amounts of LiBr (8-40% w/w) resulted in excessively hygroscopic materials that could neither be frozen solid to freeze dried. When it was possible to freeze dry the foams were unstable after exposing to atmosphere.

TABLE 25
Absorbency of psyllium foam of example 18a dried in oven-Oven at 65° C. for 7 h, then at reduced to 40° C. for 2 days.

| | | | Weight after | Weight | | | | |
|---|---|---|---|---|---|---|---|---|
| | | | liquid | after | Absorbency | | Retention | |
| | Dry weight | | absorption/ | compres- | | (W2 − | | (W3 − W1)/ |
| | W1 | Immersion | draining W2 | sion | W2 − W1 | W1)/W1 | W3 − W1 | W1 |
| Batch number | (g) | time | (g) | W3 (g) | (g) | (g/g) | (g) | (g/g) |

SOLUTION A

| | | | | | | | | |
|---|---|---|---|---|---|---|---|---|
| P-150612/PG/ | 0.2979 | 24 h | 6.2458 | 6.1540 | 5.9479 | 19.66 | 5.8561 | 19.66 |
| Tween/Ca | | | | | | | | |
| 5 cm × 5 cm; | | | | | | | | |
| ~1 mm thick | | | | | | | | |

TABLE 26
Absorbency of psyllium foam of example 18b dried in oven-Oven at 70° C. for 30 h
SOLUTION A

| | | | Weight after | Weight | | | | |
|---|---|---|---|---|---|---|---|---|
| | | | liquid | after | Absorbency | | Retention | |
| | Dry weight | Immersion | absorption/ | compres- | | (W2 − | W3 − | (W3 − W1)/ |
| | W1 | time | draining W2 | sion | W2 − W1 | W1)/W1 | W1 | W1 |
| Batch number | (g) | min | (g) | W3 (g) | (g) | (g/g) | (g) | (g/g) |
| *P-150917 /Gly /H₂O₂ | 0.2119 | 1 | 1.5669 | 1.3101 | 1.36 | 6.39 | 1.10 | 5.18 |
| (irradiated) | 0.2156 | 3 | 1.4403 | — | 1.22 | 5.68 | — | — |

*tested for haemostatic properties

For the foams of example 18a and 18b, drying in oven created a tough and dried skin on foam surface which resulted in very poor absorbency in the first hours of immersion but immersing overnight in solution A showed that foam swells reasonably in the liquid with high absorbency and excellent retention.

TABLE 27
Summary of Foam Properties for examples 1 to 18b

| Example | Batch No. | Characteristics |
|---|---|---|
| 1 | P-160330 (filtered) | White/flexible with velvet handle/crystal |
| | | appearance/compressible/1-5 mm thick and integral in both |
| | | solution A and saline |
| 2 | P- | Very white/soft/flexible/easily compressible (Ranges of |
| | 160920/Tween/H₂O₂ | thickness produced (5-10 mm). Foam integral in both solution |
| | (filtered) | A and saline but very much weaker and easily broken after |
| | | 30 min in solution |
| 3 | P- | White, soft, flexible, compressible, easily bendable but with |
| | 160928/Tween/H₂O₂ | traces of black specks. In solution, it absorbed well, held |
| | (Unfiltered) | together but less integral after storing in solution for 30 |
| | | minutes. |
| 4 | P- | The foam was soft, smooth and easily compressible where it |
| | 160923/Tween/H₂O₂ | expanded during freeze drying whilst the unexpanded section |
| | (5% psyllium) | dried into a very rough and hard foam. But in solution A and |
| | | saline, the foam was very integral even after 30 mins in |
| | | solution and absorbency increased substantially and |
| | | continuously with storage time. |
| 5 | P-160725- | Pale thin yellow dense (3-4 times denser than previous |
| | LE/Gly/H₂O₂ | examples), sticky and highly elastic foam, with the yellow |
| | Psyllium-lecithin | colour being attributed to lecithin (thickness 2 mm). Highly |
| | | integral in solution but very poor absorbency |
| 6 | P-160930/H₂O₂/ | Dull white/very flexible/compressible/non-sticky (thickness |
| | LE/PG/Ca | 4 mm) with very improved absorbency/retention in |
| | | comparison to example 5 without loss in integrity in solution |
| 7 | P- | The foam produced was dull white with reduced softness but |
| | 160310/PG/GLY/H₂O₂ | still compressible. |
| | | Broke easily on bending. Absorbency and retention were |
| | | moderate and integrity was good in the solutions irrespective |
| | | of immersion time. |
| 8 | P-160708-CS | Very pale yellow/flexible/compressible/soft/bends without |
| | Psyllium-chitosan | breaking/thickness 3-9 mm |
| | | It absorbed well within 1 minute in either solution A. In fact, |
| | | in comparison with co mmercial product, CELOX, it was |
| | | about 3 times more absorbent within 1 minute of immersion |
| | | in the liquid |
| 9 | P-161020-PE/ | The foam obtained was white, flexible, soft and easily |
| | H₂O₂/Tween 20 | compressed. Absorbency in solution A or in saline was |
| | Psyllium-pectin | immediate and substantial but with loss of integrity with time |
| | | in either the liquid |
| 10 | P-161021-PE/ | The foam obtained was white, flexible but with reduced |
| | H₂O₂/Tween 20/Ca | softness but still easily compressible. The slight surface |
| | | hardness observed had affected the foam absorbency in |
| | | comparison with example 9 and integrity only slightly |
| | | improved. |
| 11 | P-160901-GeP | The foam produced was hard, rather dense and more difficult |
| | Psyllium-gelatin | to compress. Absorbency was on the low side even after |
| | (from porcine) | immersing in solution A for 30 mins but integrity was good |
| | | throughout. |
| 12 | P-160902-GeB | The foam obtained was even harder than in example 11 and |
| | Psyllium-gelatin | more difficult to press down. Absorbency was similar to that |
| | (from bovine) | obtained for porcine gelatin and integrity but surprisingly the |
| | | foam lacked integrity especially in solution A. The |
| | | properties obtained were inferior to the co mmercial gelatin |
| | | foam, SPONGOSTAN |
| 13 | P-161205-GeB/ | The foam was soft, bent easily without crumbling and was |
| | H₂O₂/Tween 20 | more absorbent than obtained in examples 11 and 12. |
| | Psyllium-gelatin | Swelled easily in fluid but gel formed was rather weak and |
| | (from bovine) | broke easily especially after 30 minutes storage in liquid. |
| 14 | P-161212-CMC | The foam (8-10 mm thick) was soft, flexible, absorbed rapidly |
| | Psyllium-CMC | and reached almost saturation point within 1 minute of |
| | | immersion in the fluids to give a somewhat sticky gel. |
| | | Appeared that the type of CMC used may matter as |
| | | absorbency was found to increase with high viscosity grade |
| | | CMC, though integrity appeared to decrease. |
| 15 | P-161214- | Foam produced was soft and flexible with absorbency in |
| | HM/LE/PG | solution A and saline increasing with time; although initial |
| | Psyllium-High M | absorbencies in both fluids were rather low. |
| | alginate | |
| 16 | P-160804- | The foams produced were 4-5 mm thick, dense, flexible, soft |
| | HM/HG/CMC/PG/LE/ | but with rough surfaces and tough when pulled apart. |
| | Ca/Fe | Absorbency was very low (but fluid retention over 90%) in |
| | | both solution A and saline even after storing in the liquids for |
| | | 30 minutes. |
| 17 | P-170118- | Foam (2-3 mm thick) as removed from freeze dryer was hard, |
| | SF/LiBr/Tween 20 | rough and brittle but a few minutes exposure to the Lab |
| | Psyllium-silk foam | atmosphere or conditioning chamber (65% RH/20° C.) resulted |
| | | in a very flexible, soft and easy to cut foam. Foam gelled in |
| | | both saline and solution A. The gels were integral when wet |
| | | and after compression. |
| 18(a) | P- | Flexible especially when thin. Tough surface but comfortable |
| | 150612/PG/Tween/Ca | to touch. Foam was stretchy and rather sticky. |
| 18(b) | P-150917-H₂O₂/Gly | Drying in oven created a tough and dried skin on foam |
| | Oven dried) | surface which resulted in very poor absorbency in the first |
| | | hours of immersion but immersing over long period of time |
| | | such as overnight in fluids showed that foam could swell |
| | | reasonably in the liquid with high absorbency and excellent |
| | | retention |

### In-Vitro Haemostasis Testing—Clotting Time

### Assessment Method

Test pieces of the foams were cut into 1 cm×1 cm squares. 900 μl of horse blood (citrated) was pipetted into aplastic test tube in a water bath at 37° C. Once the blood had reached 37° C., 100 μl of 15 mM calcium chloride dihydrate (CaCl₂.2H₂O) was added to each tube followed by a 1 cm×1 cm sample of the test material. Each tube was shaken well and placed back in the water bath. Every 30 seconds each tube was removed from the bath, tilted and checked for clotting. Sets of samples were tested along with a blank control of blood/calcium chloride with no test sample. The time for the blood to clot was recorded and the reduction in clotting time for the sample vs. control was calculated as:

% reduction in clotting time=100%×(T_{control}−Tₛₐₘₚₗₑ)/T_{control}
where: T_{control}=clotting time for blank control
   Tₛₐₘₚₗₑ=clotting time with sample

For comparison, a commercial haemostat was tested. This product was a gelatin sponge, Spongostan™ (Johnson & Johnson Ethicon). In this test, many of the test samples showed a reduction in clotting time comparable or better than the commercial product.

### Flow/Retention Test

This testing was carried out by Quantum Management & Service GmbH, Vienna, Austria. Five (5) ml of fresh unprocessed human blood was obtained from a volunteer (no known chronic disease, no intake of any medications at least 1 month before the experiment) were obtained in a sterile syringe. One (1) ml of the fresh blood was transferred from the syringe into a 2.5 ml test tube (tube A) with a 1 cm hole on its bottom. The respective test material (test size: 2.5×2.5 cm) was placed on a test material holder with a 0.5 mm connection hose to a mechanical pump. The test material holder with the test material was placed between tube A and a second tube (tube B; 2.5 ml volume) serving as collector. The pump was started and generated a relative negative pressure (corresponding to physical suction) of −0.5 mbar during 30 seconds inside the collector. After five (5) separate measurements, new five (5) ml of fresh unprocessed human blood were obtained for the next set of measurements. Each test material was tested in total 10 times separately. The weight of the passed blood was measured and recorded in mg for each experiment. Since the initial weight of test blood was known, the difference between the initial and passed weight corresponded with the blood volume retained by the respective test materials.

Before the experiments with the test materials, exactly one (1) ml of the volunteer's blood was obtained from a blood collection tube using a calibrated pipette and transferred to an empty 1.5 ml test tube on a calibrated digital scale. The weight of 1 ml of blood was recorded in mg. The calibration was repeated 10 times. Based on the calibration measurements, 1 ml of the volunteer's blood had a weight of 1,063±2 mg. The amount of retained blood (retention %) was calculated as the mean weight of the collected blood in tube B minus the mean initial weight of the 1 ml of fresh blood in tube A, expressed in percent (%) and followed the formula:

Retention %=((mean W[V]_{A}−mean W[V]_{B})/mean W[V]_{A})×100
Where W[V]_{A}=Weight (Volume) of initial test blood in tube A
   W[V]_{B}=Weight (Volume) of passed test blood in tube B

This in-vitro test allows comparison of the performance of different test materials to retain fresh human blood passing the material with a pressure of −0.50 mbar during 30 seconds. The lower the obtained blood-weight (as surrogate for blood volume) in tube B, the higher the possible haemostatic effect of the respective test material.

### In-Vitro Haemostasis Testing—Results

The results of the in-vitro haemostasis testing for examples 1 to 17 are shown in table 28

TABLE 28
In-vitro haemostasis testing-clotting time

| | | Clotting time (mins) | | |
|---|---|---|---|---|
| | | | Control (no | % reduction in |
| Example | Sample/Batch No. | Sample | sample) | clotting time |
| 1 | P-160330 (filtered) | 18 | 30 | 40 |
| 2 | P- | 12 | 30 | 60 |
| | 160920/Tween/H₂O₂ | | | |
| | (filtered) | | | |
| 3 | P- | 13 | 30 | 57 |
| | 160928/Tween/H₂O₂ | | | |
| | (Unfiltered) | | | |
| 4 | P- | 15 | 24 | 38 |
| | 60923/Tween/ | 11 | 28 | 61 |
| | H₂O_{2 (5% psyllium)} | | | |
| 5 | P-160725- | 16 | 30 | 47 |
| | LE/Gly/H₂O₂ | | | |
| | Psyllium-lecithin | | | |
| 6 | P-160930/H₂O₂/ | >60 | 30 | N/A |
| | LE/PG/Ca | | | |
| 7 | P- | 13 | 30 | 57 |
| | 160310/PG/GLY/H₂O₂ | | | |
| 8 | P-160708-CS | 13 | 30 | 57 |
| | Psyllium-chitosan | | | |
| 9 | P-161020-PE/ | 13 | 26 | 50 |
| | H₂O₂/Tween 20 | | | |
| | Psyllium-pectin | | | |
| 10 | P-161021-PE/ | 27 | 24 | −13 |
| | H₂O₂/Tween 20/ | 31 | 28 | −11 |
| | Ca | | | |
| 11 | P-160901-GeP | 12 | 30 | 60 |
| | Psyllium-gelatin | | | |
| | (from porcine) | | | |
| 12 | P-160902-GeB | 13 | 24 | 46 |
| | Psyllium-gelatin | 11 | 28 | 61 |
| | (from bovine) | | | |
| 13 | P-161205-GeB/ | 11 | 30 | 63 |
| | H₂O₂/Tween 20 | | | |
| | Psyllium-gelatin | | | |
| | (from bovine) | | | |
| 14 | P-161212-CMC | 13 | 30 | 57 |
| | Psyllium-CMC | | | |
| 15 | P-161214- | 15 | 24 | 38 |
| | HM/LE/PG | 16 | 28 | 43 |
| | Psyllium-High M | | | |
| | alginate | | | |
| 16 | P-160804- | 15 | 24 | 38 |
| | HM/HG/CMC/PG/ | 21 | 28 | 25 |
| | LE/Ca/Fe | | | |
| 17 | P-170118- | >30 | 23 | N/A |
| | SF/LiBr/Tween 20 | | | |
| | Psyllium-silk foam | | | |
| — | Spongostan | 14 | 23 | 39 |
| | (Gelatin sponge- | | | |
| | Ethicon) | | | |

The psyllium foam of example 18b was then evaluated against several commercially available haemostat products and the results are shown below in table 28

TABLE 29
In-vitro haemostasis testing of psyllium foam of example 18b
against several commercially available haemostat products

| Material | Thickness (mm) | Blood Passed (%) | Retention (%) |
|---|---|---|---|
| Psyllium Foam | 2.5-3.0 | 7 | 93 |
| (Example 18b) | | | |
| Spongostan ™ (Gelatin | 2.5 | 50 | 50 |
| foam-Ethicon) | | | |
| Celox ™ Gauze | 1.5 | 11 | 89 |
| (chitosan coated gauze- | | | |
| Med Trade Products | | | |
| Ltd) | | | |
| Surgicel (oxidised | 1.0 | 68 | 32 |
| regenerated cellulose- | | | |
| Ethicon) | | | |
| Kaltostat ® (alginate | 1.5 | 11 | 89 |
| non-woven-Convatec) | | | |

The psyllium foam of example 18b showed the lowest passage of blood and the highest retention compared to a range of commercially available haemostat products, although differences in thickness makes direct comparison difficult except in the case of Spongostan™.

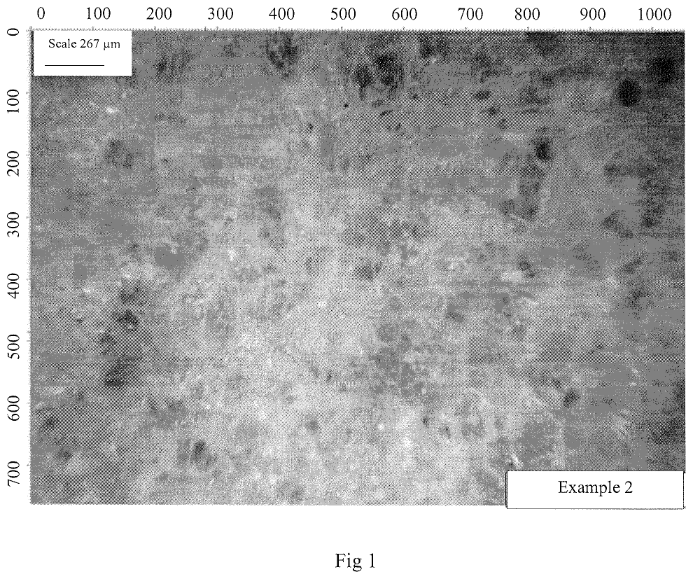

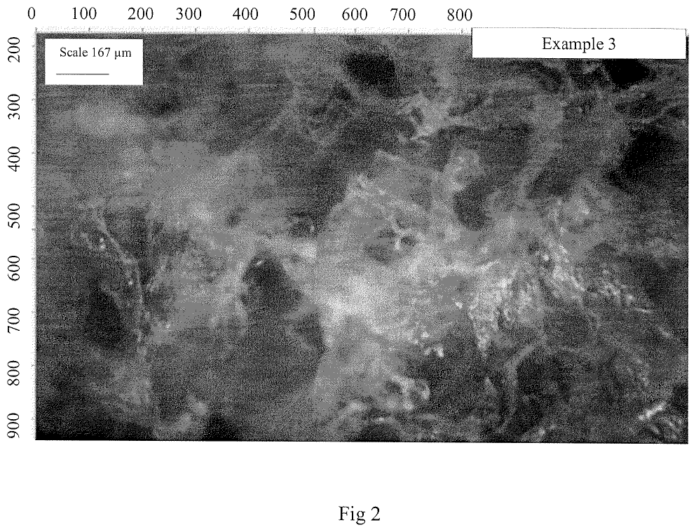

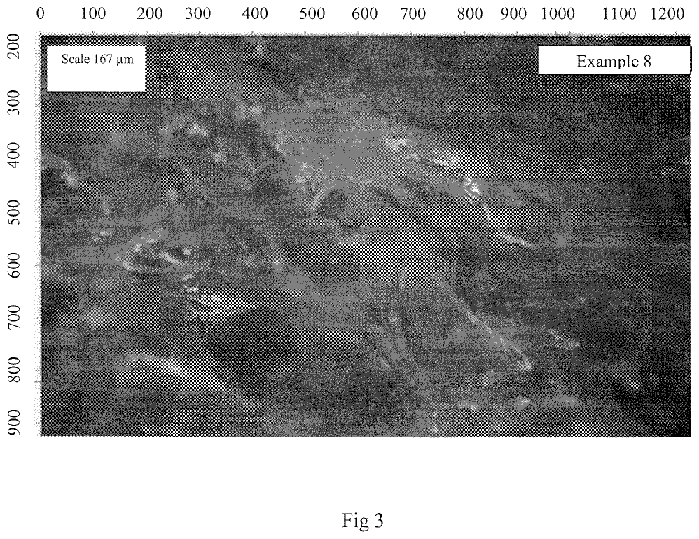

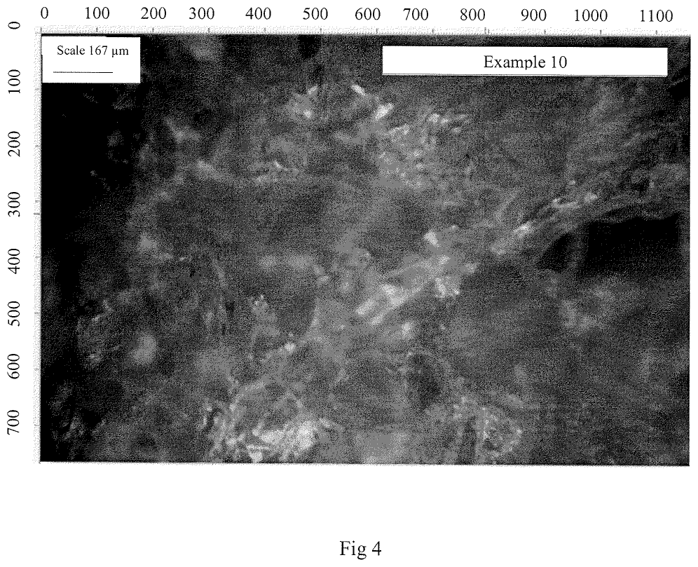

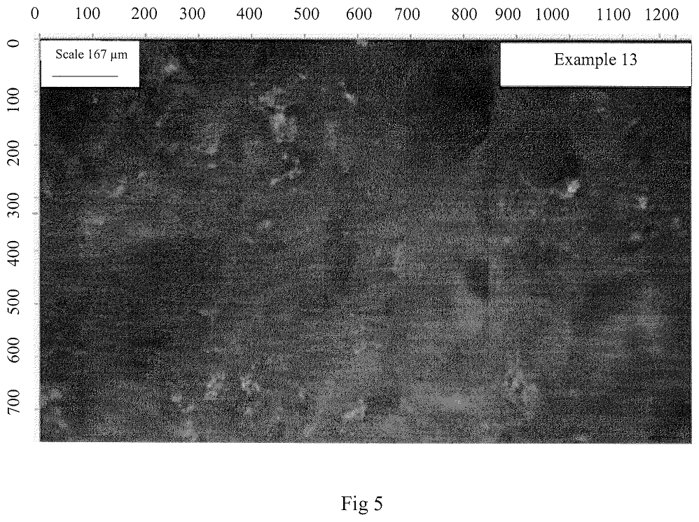

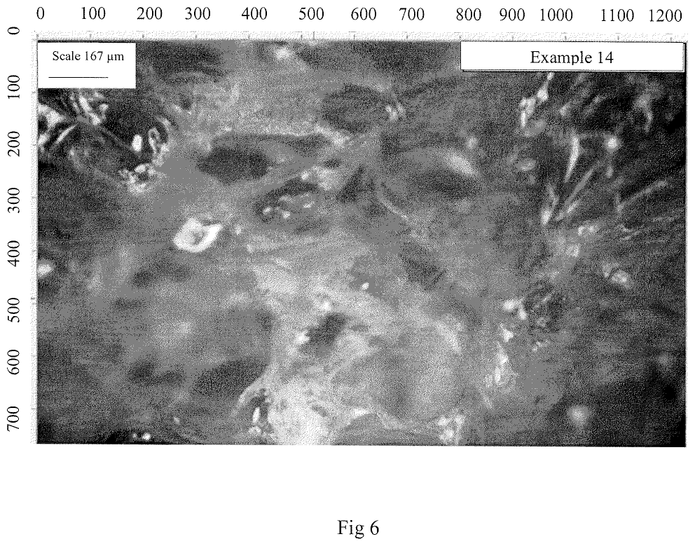

The psyllium foam structure of selected examples described above was investigated using a type XTL3T GX microscope with a trinocular head. FIG. 1 is a GX image of example 2 at 7× original magnification; FIG. 2 is a GX image of example 3 at 45× original magnification; FIG. 3 is a GX image of example 8 at 45× original magnification; FIG. 4 is a GX image of example 10 at 45× original magnification; FIG. 5 is a GX image of example 13 at 45× original magnification; FIG. 6 is a GX image of example 14 at 45× original magnification.

It is noted from the microscope images of FIGS. 1 to 6 that pore shape and size appears irregular within each foam structure. Additionally, each foam structure appears to include what could be considered a tunnelling network forming an open matrix and not discreet isolated circular pores. This open pore network or matrix microstructure is believed to be advantageous for the control of moisture and vapour transmission across the material, the wicking characteristics and further the haemostatic characteristics of the material.

1. A moisture absorbent material comprising:
a psyllium foam.

2. The material as claimed in claim 1 wherein the psyllium foam is compositionally a main component of the material by wt %.

3. The material as claimed in claim 1 comprising psyllium foam in at least 50 wt %, at least 60 wt %, at least 70 wt %, at least 80 wt %, at least 90 wt % or at least 95 wt %.

4. The material as claimed in claim 1 wherein the moisture absorbent material is porous comprising a pore size in a range 50 μm to 2 mm; 100 μm to 2 mm; 200 μm to 2 mm; 300 μm to 2 mm; 400 μm to 2 mm; 500 μm to 2 mm; 600 μm to 2 mm; 700 μm to 2 mm; 800 μm to 2 mm; 50 μm to 1.5 mm; 50 μm to 1 mm; 50 μm to 800 μm; 100 μm to 800 μm; 200 μm to 800 μm; 300 μm to 800 μm; 400 μm to 800 μm; 50 μm to 600 μm; or 200 μm to 600 μm.

5. The material as claimed in claim 1 wherein the psyllium foam is derived from psyllium husk.

6. The material as claimed in claim 1 wherein the psyllium foam is derived from any one or a combination of the species:
Plantago Ovata
Plantago major
P. psyllium
P. amplexicauli
P. asiatica
P. lanceolate
P. insularis.

7. The material as claimed in claim 1 comprising a porosity of 50% to 99.5%.

8. The material as claimed in claim 1 further comprising any one or a combination of:
hydrogen peroxide;
a surfactant;
lecithin.

9. The material as claimed in claim 1 further comprising any one or a combination of:
a polysaccharide
a polysaccharide based material
a hydrocolloid forming compound
an alginate
chitosan
chitin
pectin
carboxymethyl cellulose
hydroxylpropyl methylcellulose
gellan
konjac.

10. The material as claimed in claim 1 further comprising any one or a combination of:
a protein
silk
gelatin
collagen
keratin.

11. The material as claimed in claim 1 further comprising any one or a combination of:
a synthetic polymer
polyethylene oxide
polyacrylic acid
acetic acid
polyacrylate
polyacryolnitrile
polyvinyl alcohol
a polyol
glycerol
a glycol
a diol
an alkaline glycol
propylene glycol.

12. The material as claimed in claim 1 further comprising any one or a combination of:
calcium chloride hydrate
calcium chloride dihydrate
ferric sulphate hydrate
ferric sulphate dehydrate.

13. The material as claimed in claim 1 further comprising any one or a combination of:
a drug
an enzyme
a growth enhancer
a living cell
an antimicrobial
a metal based antimicrobial
an antimicrobial agent metal ion selected from any one or a combination of: Ag, Zn, Cu, Ti, Pt, Pd, Bi, Sn, Sb.

14. A wound dressing comprising a material as claimed in claim 1.

15. A haemostat dressing comprising a material as claimed in claim 1.

16. A hygiene article comprising a material as claimed in claim 1.

17. A mammalian sanitary pad comprising a material as claimed in claim 1.

18. A liquid filtration medium comprising a material as claimed claim 1.

19. A food packaging comprising a material as claimed in claim 1.

20. A method of manufacturing a moisture absorbent material comprising:
forming a psyllium gel from an aqueous psyllium solution;
creating a psyllium foam from the psyllium gel.

21. The method as claimed in claim 20 wherein the step of creating the psyllium foam from the psyllium gel comprises freeze drying the psyllium gel.

22. The method as claimed in claim 20 wherein the step of creating the psyllium foam from the psyllium gel comprises agitating the psyllium gel to create air bubbles to form the psyllium foam and drying the psyllium foam.

23. The method as claimed in claim 20 wherein the step of creating the psyllium foam from the psyllium gel comprises adding a foaming agent to the psyllium solution and/or psyllium gel and drying the psyllium foam.

24. The method as claimed in claim 23 wherein the foaming agent comprises any one of a combination of the following:
hydrogen peroxide;
a surfactant;
lecithin.

25. The material as claimed in claim 20 further comprising irradiating the foam.

26. The method as claimed in claim 20 further comprising adding any one or a combination of the following
a polysaccharide;
a polysaccharide based material;
a hydrocolloid forming compound;
an alginate;
chitosan;
chitin;
pectin;
carboxymethyl cellulose;
hydroxylpropyl methylcellulose;
gellan;
konjac
to the psyllium solution or psyllium gel.

27. The method as claimed in claim 20 further comprising adding any one or a combination of the following
a protein;
silk;
gelatin;
collagen;
keratin
to the psyllium solution or psyllium gel.

28. The method as claimed in claim 20 further comprising adding any one or a combination of the following
a synthetic polymer;
polyethylene oxide;
polyacrylic acid;
acetic acid;
polyacrylate;
polyacryolnitrile;
polyvinyl alcohol
to the psyllium solution or psyllium gel.

29. The method as claimed in claim 20 further comprising adding any one or a combination of the following
hydrogen peroxide;
a surfactant;
lecithin
to the psyllium solution or psyllium gel.

30. The method as claimed in claim 20 further comprising adding any one or a combination of the following
calcium chloride hydrate;
calcium chloride dihydrate;
ferric sulphate hydrate;
ferric sulphate dihydrate;
to the psyllium solution or psyllium gel.

31. The method as claimed in claim 20 further comprising adding any one or a combination of the following
a drug;
an enzyme;
a growth enhancer;
a living cell;
an antimicrobial;
a metal based antimicrobial;
an antimicrobial agent metal ion selected from any one or a combination of: Ag, Zn, Cu, Ti, Pt, Pd, Bi, Sn, Sb
to the psyllium solution or psyllium gel.
